SEQUENCE LISTING (12 sequences)

SEQ ID NO: 1 - DNA, 25 bp, Artificial Sequence
  Synthetic Construct
ccaggtatggctgccgatggttatc

SEQ ID NO: 2 - DNA, 30 bp, Artificial Sequence
  Synthetic Construct
gtccaatcgatgcgaagcgcaaccaagcag

SEQ ID NO: 3 - DNA, 25 bp, Artificial Sequence
  Synthetic Construct
ccaggtatggctgctgacggttatc

SEQ ID NO: 4 - DNA, 36 bp, Artificial Sequence
  Synthetic Construct
gtccaatcgatgcagttgtaaaccgcgaagcgcaag

SEQ ID NO: 5 - DNA, 26 bp, Artificial Sequence
  Synthetic Construct
ccagatatgactgacggttaccttcc

SEQ ID NO: 6 - DNA, 36 bp, Artificial Sequence; identical to SEQ ID NO: 4
  Synthetic Construct

SEQ ID NO: 7 - DNA, 26 bp, Artificial Sequence
  Synthetic Construct
cactgacgtcaatgtgacgtcctagg

SEQ ID NO: 8 - DNA, 25 bp, Artificial Sequence
  Synthetic Construct
cgtgacctccttgacctggatgttg

SEQ ID NO: 9 - DNA, 21 bp, Artificial Sequence
  Synthetic Construct
ggaaaacttgtcagattttgg

SEQ ID NO: 10 - DNA, 37 bp, Artificial Sequence
  Synthetic Construct
gtccaatcgatgccacaagaggcagtattttactgac

SEQ ID NO: 11 - DNA, 22 bp, Artificial Sequence
  Synthetic Construct
ctggatgactgtgtttctgagc

SEQ ID NO: 12 - DNA, 30 bp, Artificial Sequence
  Synthetic Construct
gtccaatcgatgcgaagcgcaactaagcag

(19) United States
(12) Patent Application Publication
(10) Pub. No.: US 2004/0235174 A1
(43) Pub. Date: Nov. 25, 2004

(54) Aav helper plasmids for helper virus-free packaging and pseudo typification of aav vectors

(76) Inventors: Dirk Grimm, Ludwigshafen (DE); Jurgen Kleinschmidt, Bammental (DE)

Correspondence Address: Steven J Hultquist, Intellectual Property/Technology Law, P O Box 14329, Research Triangle Park, NC, 27709, US

(21) Appl. No.: 10/475,931

(22) PCT Filed: Apr. 24, 2002
(86) PCT No.: PCT/DE02/01502

(30) Foreign Application Priority Data
Apr. 25, 2001 (DE) 101 20 265.2

(51) Int. Cl. C12N 15/861

(52) U.S. Cl. 435/457

(57) ABSTRACT

The invention relates to AAV helper plasmids for the helper virus-free packaging of AAV vectors. These AAV helper plasmids comprise the following DNA sequences: (a1) the rep gene of AAV-2, and (a2) the cap gene of AAV-1, AAV-3, AAV-4, AAV-5 or AAV-6, or (b) the cap gene and the rep gene of AAV-1, AAV-3, AAV-4, AAV-5 or AAV-6 each, and (c) all of the other helper virus DNA sequences necessary for forming AAV particles. The invention also relates to the use of these AAV helper plasmids or AAV particles with a coat encoded by these AAV helper plasmids and an AAV expression vector for gene therapy.

The present invention relates to AAV helper plasmids for the helper virus-free packaging and pseudotyping of AAV vectors. These AAV helper plasmids comprise the following DNA sequences: (a1) The rep gene of AAV-2 and (a2) the cap gene of AAV-1, AAV-3, AAV-4, AAV-5 or AAV-6, or (b) the cap gene and the rep gene of AAV-1, AAV-3, AAV-4, AAV-5 and AAV-6 each, and (c) all further helper virus DNA sequences necessary for forming AAV particles. The invention also relates to the use of these AAV helper plasmids and AAV particles having a coat encoded by these AAV helper plasmids and an AAV expression vector for gene therapy.

AAVs are single-stranded DNA viruses belonging to the parvovirus family. For replication AAVs require helper viruses, in particular adenoviruses or herpes viruses. In the absence of helper viruses, AAVs integrate into the host cell genome, in particular at a specific site of chromosome 19. The genome of AAVs is linear and has a length of about 4680 nucleotides. It comprises two reading frames coding for a structural gene and a non-structural gene. The structural gene is referred to as a cap gene. It is controlled by the P40 promoter and codes for three capsid proteins. The non-structural gene is referred to as a rep gene and codes for the Rep proteins Rep 78, Rep 68, Rep 52 and Rep 40. The two former ones are expressed under the control of the P5 promoter, while the expression of Rep 52 and Rep 40 is controlled by the P19 promoter. The functions of the Rep proteins are inter alia the regulation of replication and transcription of the AAV genome.

AAVs have been developed and tested for intensively as possible vectors for human gene therapy for some time now. Among the six different AAV serotypes (AAV-1 to AAV-6) which have been cloned and sequenced to date, AAV-2 is the best characterized serotype and most of the vectors used for the time being are based on AAV-2. However, reports on the production and evaluation of the other five AAV serotypes have also been published in the past few years. It turned out that the ITRs (inverted terminal repeats) at either end of the AAV genome are the only cis elements required for proliferation (i.e. excision of the viral DNA from the plasmid, replication and packaging of the intermediary DNA sequences) of AAV vectors (by means of the helper virus). It was thus suggested that, in principle, any DNA flanked by the AAV-ITRs and comparable with the wild-type virus genome as regards length, can be packed into AAV capsids in the presence of the rep and cap gene products in trans and the helper virus functions. In most cases, a method was used for the production of these vectors in which helper viruses have to be used, i.e. the cells are cotransfected with the AAV vector and helper plasmids and then infected with the helper adenovirus, which results in recombinant AAV vectors contaminated with adenovirus though. Another strategy is based on a triple transfection in which a non-infectious adenoviral plasmid is additionally used to avoid a contamination by means of helper viruses so as to provide helper functions.

In summary, there are presently three different approaches: (a) cotransfection of AAV helper sequences providing the rep and cap genes of the respective AAV serotype, and the corresponding vector plasmid of AAV-2, AAV-3, AAV-5 or AAV-6, (b) cotransfection of AAV-2 vector plasmids with AAV helper plasmids carrying the rep gene of AAV-2 and the cap gene of AAV-1, AAV-3 or AAV-5, and (c) cotransfection of AAV-2 vector plasmids with rep-cap genes of AAV-1, AAV-3, AAV-4 or AAV-6. In all of the three approaches, the adenoviral helper functions are provided by infection with adenoviruses or by additional transfection of plasmids carrying the adenoviral genome. However, all of the former approaches have certain serious drawbacks. For example, (a) either different vector plasmids have, to be used for the packaging into different AAV serotypes, (b) the vector production by triple infection is complicated and expensive, and (c) the double transfection and infection with adenoviruses results in the problem of contamination with adenoviruses.

The technical problem underlying the present invention is thus to provide a method of packaging AAV vectors which do not have the above discussed drawbacks, i.e. permit helper virus-free packaging of AAV vector DNA into a desired AAV capsid by simple cotransfection with a suitable helper/packaging plasmid, no contamination with adenovirsues occurring.

This technical problem is solved by providing the embodiments characterized in the claims. It has been found surprisingly that it was possible to solve the technical problem by using a helper plasmid permitting the complete helper functions for the packaging of the vector plasmid derived from AAV, preferably AAV-2, into the desired AAV capsid. The main advantage is here the simplification of the production of pseudo-typed AAV vectors which are also free of adenovirus contamination. In this simple method, the pDG helper plasmid described in German patent application 196 44 500.0-41 was used as a basis, which includes all of the AAV-2 and adenoviral genes whose products are necessary for the production of AAV-2 vectors. For this purpose, the cap gene of AAV serotype 2 on this plasmid was substituted for a cap gene of serotype 1, 3, 4, 5 or 6, a total of five new helper plasmids being obtained which are designated as pDP1, pDP3, pDP4, pDP5 and pDP6, respectively. In the case of pDP4, it proved to be particularly favorable to also substitute the rep gene of AAV-2 for the rep gene of AAV-4. The cotransfection of a AAV-2 vector plasmid with the respective helper plasmid yielded recombinant AAV particles consisting of the AAV-2 vector which corresponded in AAV capsid coats according to the serotype of the employed pDP-derived helper plasmid. The different vector parent solutions were analyzed as regards the titers with fully assembled infectious particles containing DNA and the different efficiencies of the vector production were compared. Here, it should be noted that all of the recombinant AAV parent solutions were free of contaminations with wild-type AAV. In addition, an expression cassette can be inserted in every helper plasmid, which contains e.g. the gene for the red fluorescent protein (“Dsred”, Clontech, Palo Alto, U.S.A.) under the control of the RSV promoter and following excitation with a suitable wavelength the successfully transfected cells can thus easily and readily be identified by means of the bright red color.

The subject matter of the present invention is thus an AAV helper plasmid comprising the following DNA sequences:

(a1) the rep gene of AAV-2; and

(a2) the cap gene of AAV-1, AAV-3, AAV-4, AAV-5 or AAV-6; or

(b) the cap gene and the rep gene of AAV-1, AAV-3, AAV-4, AAV-5 or AAV-6 each; and

(c) all of the other helper virus DNA sequences necessary for forming AAV particles.

The term “helper virus DNA sequences” used herein relates to all the DNA sequences of a helper virus necessary to produce AAV particles. Such DNA sequences preferably originate from herpes and/or adenoviruses, most preferably from adenovirus 5. The sequences may comprise the entire virus genome or fragments thereof. Suitable helper virus DNA sequences are described as starting material for the production of the AAV helper plasmids according to the invention in German patent application 196 44 500.0-41, for example, and also comprise the DNA sequences disclosed in this patent application of the plasmid pTG 9585, which as the helper virus DNA sequence comprises the entire adenovirus 5 sequence with the exception of the E1 region. The AAV helper plasmid according to the invention may also contain helper virus DNA sequences differing from those in pTG 9585 in that they have a deletion in the structural gene L1 of the Ad5 sequence, in particular in the region of nucleotides 16614-18669.

The term “AAV helper plasmid” used herein does not only relate to helper plasmids with the genes listed originally under items (a) to (c) but also to helper plasmids with modified genes which include deletions or insertions of nucleotides, for example, but still code for proteins having the desired biological function. The person skilled in the art can determine by means of common methods whether a modified gene still codes for a product having the desired biological function. The person skilled in the art is also familiar with sources for the individual genes distinguishing the AAV helper plasmid according to the invention. General methods known in the art can be used for the construction of AAV helper plasmids containing the above DNA sequences and optionally further sequences. These methods comprise e.g. in vitro recombination techniques, synthetic methods and in vivo recombination methods as described inter alia in Sambrook et al., Molecular Cloning: A Laboratory Manual, 2^{nd} edition, Cold Spring Harbor Laboratory Press, Cold Spring Harbor N.Y. (1989). For example, the pDG plasmid described in German patent application 196 44 500.0-41 can be used as a basic scaffold for a helper plasmid according to the invention. In said plasmid, the original AAV-2 cap gene is substituted for a cap gene of AAV-1, AAV-3, AAV-4, AAV-5 or AAV-6 (AAV-1, Xiao et al., J. Virol. 73 (1999), 3994-4003; AAV-3, Muramatsu et al ., Virol. 221 (1996), 208-217; AAV-4, Chiorini et al., J. Virol. (1997), 6923-6833; AAV-5, Bantel-Schaal et al., J; Virol. 73 (1999), 939-947; Chiorini et al., J. Virol. 73 (1999), 1303-1319; AAV-6, Rutledge et al., J. Virol. 72 (1998), 309-319) via PCR and suitable primers, for example.

In a preferred embodiment, the AAV helper plasmid according to the invention contains as helper virus DNA sequences the Ad5 genes E2A, E4 and VA, which may be derived from the pDG plasmid described in the German patent application 196 44 500.0-41, for example, and which are controlled by the respective original promoter or are controlled by heterologous promoters.

In a particularly preferred embodiment, the AAV helper plasmid according to the invention additionally contains an expression cassette for the expression of a marker protein, preferably a fluorescent protein. In this connection, the term “expression cassette” means a combination of a gene coding for a fluorescent gene, for example, which is controlled by a suitable promoter. This permits the simple detection of a transfection of the desired target cell. Examples of genes coding for suitable fluorescent proteins are rfp-(red), gfp-(green), cfp-(cyan), yfg-(yellow) and luciferase-coding gene and examples of suitable promoters are RSV (Rous sarcoma virus) promoter, CMV (cytomegalovirus) promoter and HSU (herpes simplex virus)-tk promoter. This expression cassette is inserted in the AAV helper plasmid, preferably in the ClaI cleavage site between the end of the cap gene and the beginning of the adenoviral VA gene. This ClaI cleavage site is present in pDG and in pDP1, pDP3, pDP4, pDP5 and pDP6.

The following AAV helper plasmids were deposited with the DSMZ [German-type collection of micro-organisms and cell cultures], Braunschweig, Germany, in accordance with the provisions of the Budapest Treaty on April 23, 2001: pDP1 with accession number DSM 14256, pDP3 with accession number DSM 14255, pDP4 with accession number DSM 14254, pDP5 with accession number DSM 14253, and pDP6 with accession number DSM 14252.

The subject matter of the present invention also relates to AAV particles whose capsid coat is encoded by an AAV helper plasmid according to the invention and which contains an AAV expression vector. Suitable AAV expression vectors are known to the person skilled in the art (Zolotukhin et al., J. Virol. 70 (1996), 4646-4653). This AAV expression vector is preferably an expression vector which comprises at least the following DNA sequences: (a) the 5′ITR and 3′ITR of an AAV-2; (b) a constitutive or inducible promoter active in mammals, and (c) a polyadenylation signal. Here, the terms “5′ITR” and “3′ITR” comprise all of the 5′ITR” and 3′ITR” sequences permitting the integration of the vector into the host genome. The herein used term “a constitutive or inducible promoter active in mammals” comprises all of the promoters which in mammals permit the transcription of the desired DNA sequence, above all those resulting in an intense expression, preferably heterologous promoters. Suitable promoters are known to the person skilled in the art and comprise e.g. the constitutive promoters CMV and cytokeratin K14 promoters or the inducible promoters MMTV (mouse mammary tumor virus), metallothionein and promoter systems which can be regulated by tetracycline (Tet-on/-off). The AAV expression vector can also contain the desired gene to be expressed in the mammalian cells whose expression is desired for a gene therapy, for example. In addition, the AAV expression vector may contain a gene which codes for a detectable phenotypic marker, proving the successful introduction of the AAV expression vector into the target cell. Suitable marker genes are the above-mentioned ones, for example.

AAV particles according to the invention can be obtained by suitable methods, e.g. by cotransfection of mammalian cells, e.g. COS cells or 293 cells, with an AAV helper plasmid according to the invention and an above-described AAV expression vector, e.g. by means of the method described in below Example 2. The titer obtainable is usually between 10⁶ and 10⁸ viral particles/ml.

A gene therapy can be carried out with an AAV helper plasmid or AAV particle according to the invention, the cells being transfected by common methods. For example, electroporation, lipofection and preferably calciumphosphate precipitation are to be mentioned as transfection techniques. The cells may be available in an organism, and the cells to be transfected can also be isolated from an organism, be transfected outside the organism and then be returned to the organism again. Such cells are referred to as autologous cells. In addition, as regards the organism it is also possible to use allogenic cells for transduction. Here, it is favorable for these cells to belong to an HLA type corresponding to the organism. The person skilled in the art is familiar with methods of giving the cells a certain HLA type.

The present invention also relates to the above described AAV helper plasmids and host cells containing AAV expression vectors, which may serve for producing and collecting AAV particles, for example. These host cells comprise mammalian cells, preferably 293, 911 or PerC6 cells. Methods for the transfection these host cells, for the phenotypic selection of transfectants, etc., are known to the person skilled in the art. The person skilled in the art is also familiar with suitable culturing methods and media to be able to culture mammalian cells. The culture medium may be any medium usually used for culturing mammalian cells, e.g. IMEM, DMEM, etc. The cells are cultured in the above medium under suitable conditions, optionally with (partially) renewing the medium at suitable intervals. Suitable conditions, e.g. as regards suitable containers, temperature, relative humidity, O₂ content and CO₂ content of the gas phase are known to the person skilled in the art. The cells are preferably cultured in the above medium under the following conditions: (a) 37° C., (b) 100% relative humidity, (c) 10% O₂ and (d) 5% to 7% CO₂. The AAV particles, preferably from the culture supernatant, can be collected by common standard methods, e.g. freeze-thaw lysis, filtration, centrifugation and chromatographic separation and concentration. It is preferred to further purify the AAV particles, above all for clinical applications, e.g. via ion exchange chromatography and heparin affinity chromatography.

The subject matter of the present invention also relates to a medicament containing an AAV helper plasmid or AAV particle according to the invention. Here, the medicament may additionally contain a pharmaceutical compatible carrier. Suitable carriers and the formulation of such medicaments are known to the person skilled in the art. Suitable carriers are e.g. phosphate-buffered saline solutions, water, emulsions, e.g. oil/water emulsions, wetting agents, sterile solutions, etc. The kind of carrier depends on how to administer the AAV helper plasmid or AAV particle according to the invention. The suitable dosage is determined by the attending physician and depends on various factors, e.g. on the patient's age, sex and weight, the severity of the disease, the kind of administration, etc. In this connection, it turned out that high transduction rates can be achieved with the most different cells, e.g. primary cells of the corneal epithelium or muscle cells.

### BRIEF DESCRIPTION OF THE FIGURES

FIG. 1: PCR strategy for producing the AAV helper plasmids pDP1 and pDP3 to pDP6

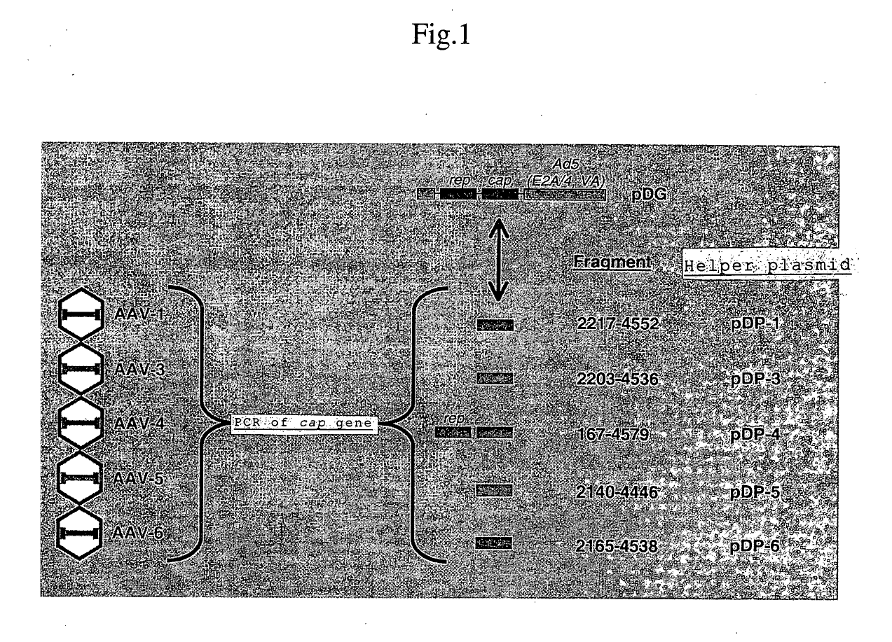

The numerical values indicated in the “fragment” column refer to the first and last base pairs contained in the respective cap (or rep and cap genes). The values here relate to the genome of the respective serotype.

FIG. 2: Physical map of the AAV helper plasmid pDP1 Positions 483-1293: MMTV-LTR, 1293-3218: rep gene of AAV-2, 3219-5555: cap gene of AAV-1, 5558-7256: VA, 12890-8218 (C) (C=complementarily opposed): E2A, 13407-14784: *E3* (*E3*=rest of the E3 region), 19503-16691 (C): E4.

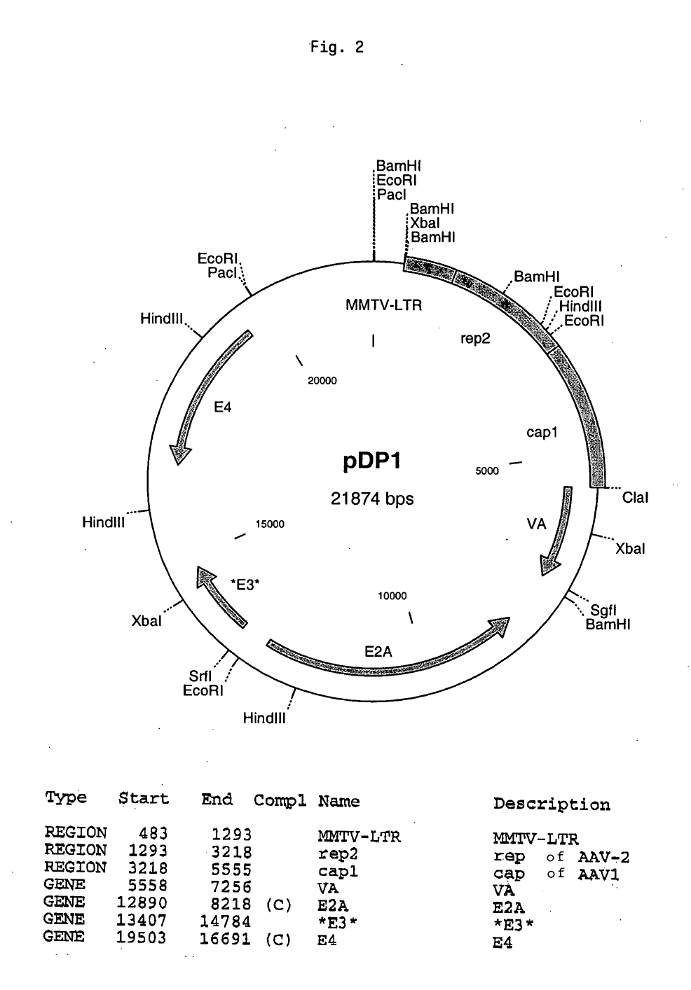

FIG. 3: Physical map of the AAV helper plasmid pDP3 Positions 483-1293: MMTV-LTR, 1294-3218: rep gene of AAV-2, 3219-5577: cap gene of AAV-3, 5558-7276: VA, 12910-8238 (C): E2A 13427-14804: *E3*, 19523-16711 (C): E4.

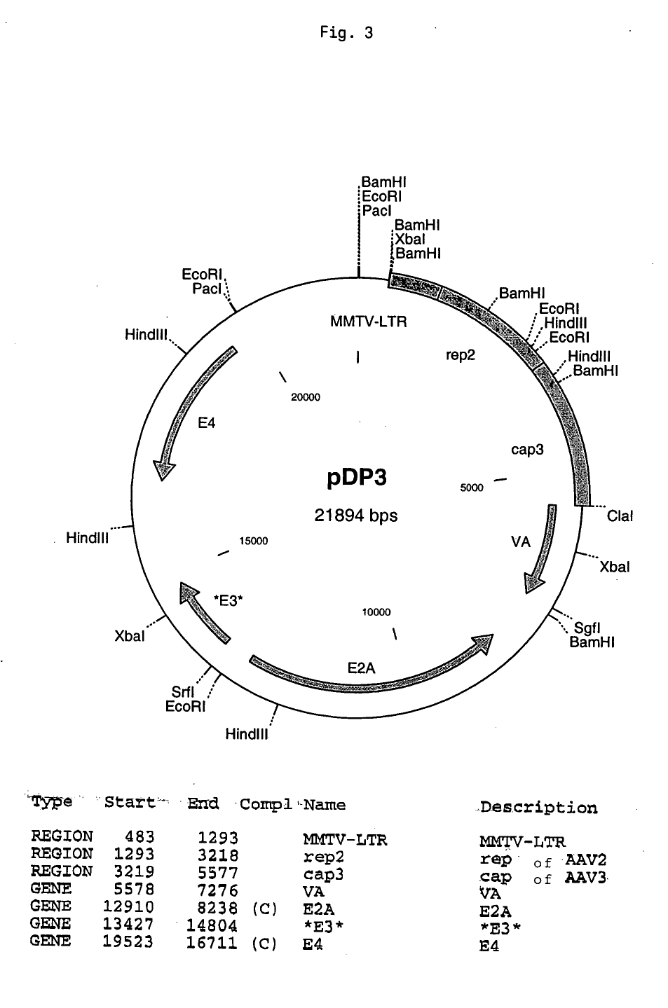

FIG. 4: Physical map of the AAV helper plasmid pDP4 Positions 483-1293: MMTV-LTR, 1293-3218: rep/cap gene, 5573-7271: VA, 7880-3219: VA, 12905-8233 (C) : E2A, 13422-14799: *E3*, 19518-16706 (C): E4.

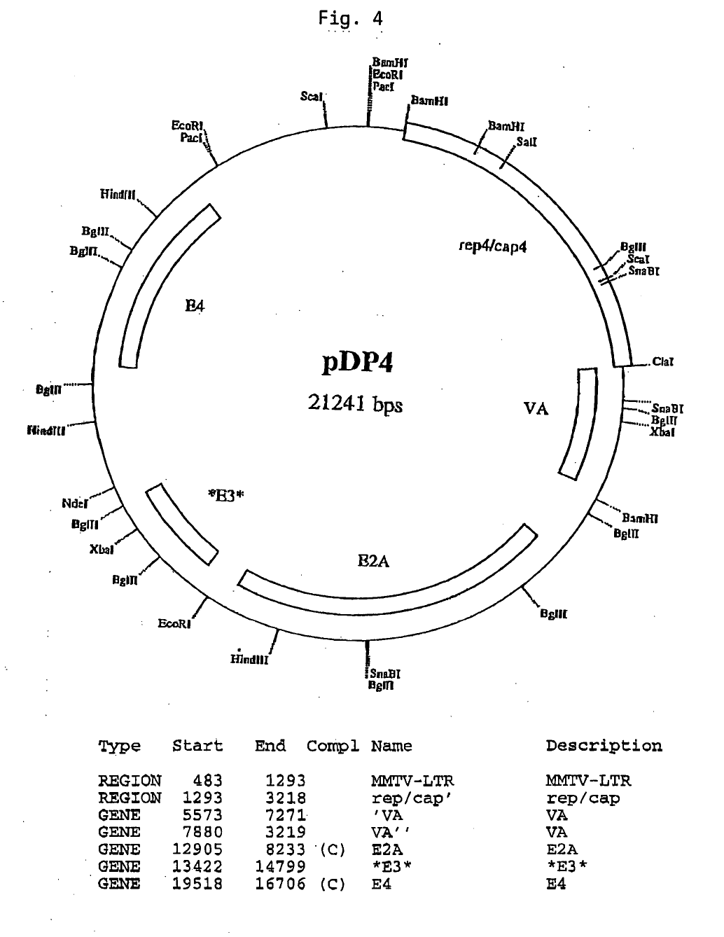

FIG. 5: Physical map of the AAV helper plasmid pDP5 Positions 483-1293: MMTV-LTR, 1294-3218: rep gene of AAV-2, 3219-5518: cap gene of AAV-5, 5519-7217: VA, 12851-8179 (C): E2A, 13368-14745: *E3*, 19464-16652 (C): E4.

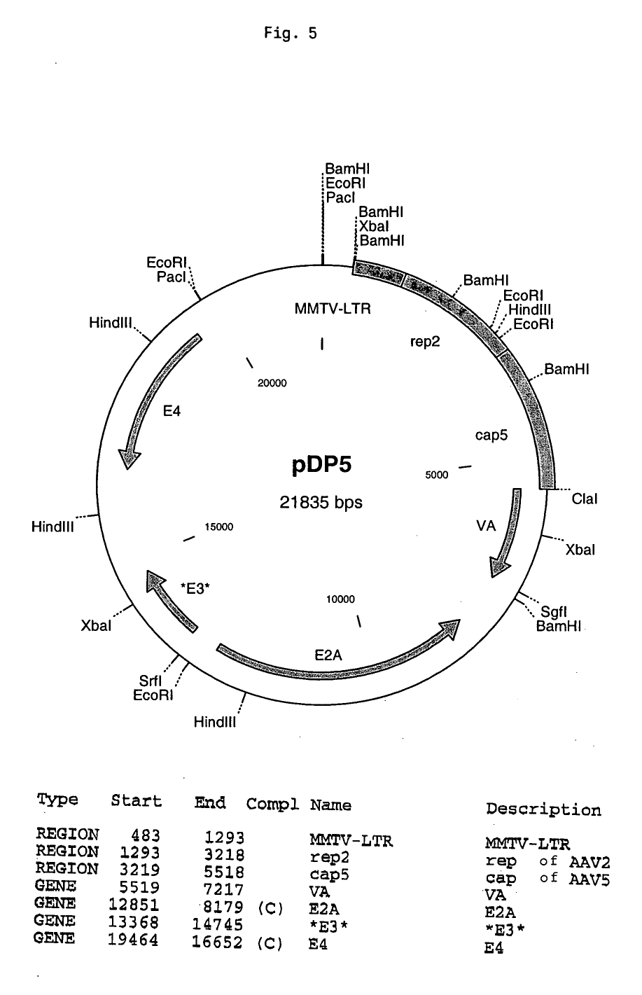

FIG. 6: Physical map of the AAV helper plasmid pDP6 Positions 483-1293: MMTV-LTR, 1294-3218: rep gene of AAV-2, 3219-5598: cap gene of AAV-3, 5596-7294: VA, 12928-8256 (C):. E2A, 13445-14822: *E3*, 19541-16729 (C): E4.

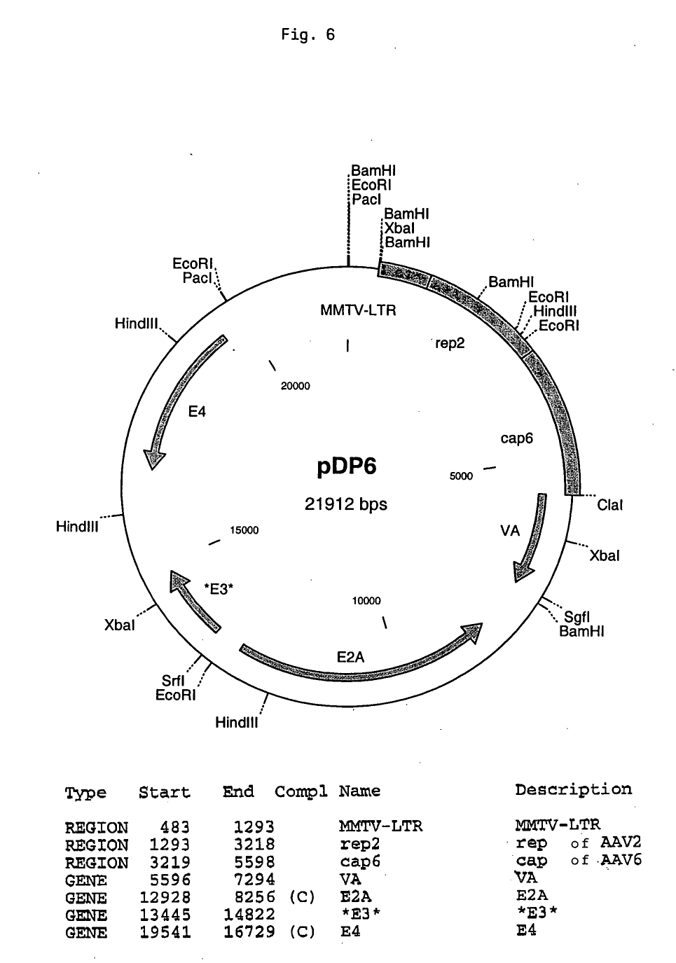

FIG. 7: Production of pseudotyped AAV-2 vectors

The top portion of the figure shows by way of diagram the AAV-2 vectors packed for titration into different capsids (Grimm et al., Gene Therapy, 6 (1999), 1322-1330).

The bottom portion of the figure shows representative titers of AAV-2 vectors packed by the described method into capsids of AAV-2 or into capsids of AAV-1, AAV-3, AAV-4, AAV-5 or AAV-6.

The invention is explained by the below examples.

### EXAMPLE 1

### Production of the AAV Helper Plasmids pDP1 and pDP3 to pDP6

For the production of the AAV helper plasmids pDP1 and pDP3 to pDP6, the pDG plasmid described in German patent application 196 44 500.0-41 was used as a basis. It has a total length of 21846 base pairs. Together with the MMTV promoter substituting the AAV-2 p5 promoter the AAV genome contained in pDG has a total length of 5044 base pairs.

First, the 293 cells were infected with the different serotypes and Ad-5 as a helper virus under standard conditions. After three days, replicated viral AAV-DNA was isolated from the cells and purified. This DNA then served as a template in a PCR reaction for amplification of the cap genes of the. respective serotypes (Table 1). In the case of AAV-4 for the production of pDP4, the rep gene was also amplified. The primers for the different PCR reactions were in this connection chosen such that at the 3′ end of the respective products a cleavage site for the restriction enzyme ClaI was obtained, however, the left end was blunt (i.e. without cleavage site or overhanging end). For cloning the PCR fragments, the pDG plasmid was linearized with the enzymes SwaI (cleaving without overhanging ends) and ClaI so as to remove the cap AAV-2 gene originally contained in the pDG plasmid. The respective cap genes of the other AAV serotypes were then cloned thereinto instead. In the case of AAV-4, the rep and cap genes were amplified. At the left end of the rep gene, a BlnI recognition site was amplified from the AAV-4 genome. After a corresponding restriction, this permitted the cloning of the AAV-4 rep gene together with the AAV-4 cap gene (cleavage with ClaI taking place again at the 3′ end as described above) into the pDG plasmid cleaved with XbaI (compatible with BlnI) and ClaI. Both the rep and cap genes of AAV-2 are removed from the pDG plasmid by digestion with XbaI and ClaI.

### EXAMPLE 2

### Production of Pseudotyped AAV-2 Vectors

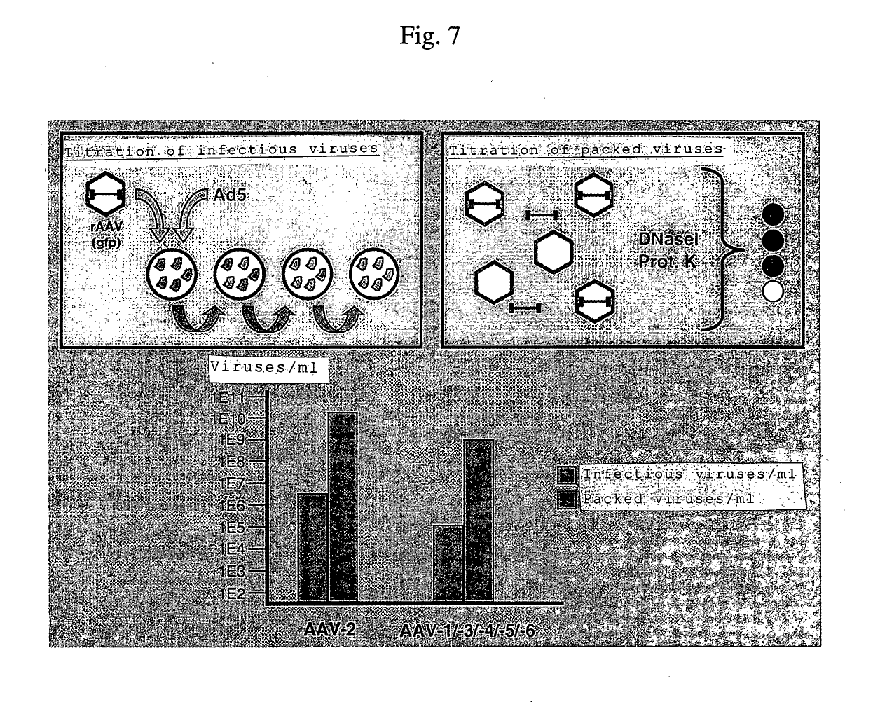

For the production (shown by way of diagram in FIG. 7) of pseudotyped AAV-2 vectors 293T cells were cotransfected with the AAV-2 vector plasmid pTRUF5 (Zolotukhin et al., J. Virol. 70 (1996), 4646-4654) and one of the 6 different AAV helper plasmids (pDG, pDP1, pDP3, pDP4, pDP5 and pDP6) each, i.e. in all of the cases the vector plasmid used was identical and only the AAV helper plasmid varied. After three days of incubation, crude supernatants from the cells were collected by freeze/thaw lysates and the resulting viruses were quantified. A supernatant from cells cotransfected with a vector and an AAV helper plasmid according to the invention typically contains between 10⁶ and 10⁷ infectious particles per ml. The infectious viruses or packed genomes were titrated according to the method described in Grimm et al., Gene Therapy 6 (1999), 1322-1330.

TABLE 1

Oligonucleotides used for the
amplification of the AAV cap (and AAV-4 rep) genes

| | | |
|---|---|---|
| AAV-1: | (left) | 5′-CCAGGTATGGCTGCCGATG |
| | | GTTATC-3′ |
| | (right) | 5′-GTCCAATCGATGCGAAGCG |
| | | CAACCAAGCAG-3′ |
| AAV-3: | (left) | 5′-CCAGGTATGGCTGCTGACG |
| | | GTTATC-3′ |
| | (right) | 5′-GTCCAATCGATGCAGTTGT |
| | | AAACCGCGAAGCGCAAG-3′ |
| AAV-4: | (cap, left) | 5′-CCAGATATGACTGACGGTT |
| | | ACCTTCC-3′ |
| | (cap, right) | 5′-GTCCAATCGATGCAGTTGT |
| | | AAACCGCGAAGCGCAAG-3′ |
| | (rep, left) | 5′-CACTGACGTCAATGTGACG |
| | | TCCTAGG-3′ |
| | (rep, right) | 5′-CGTGACCTCCTTGACCTGG |
| | | ATGTTG-3′ |
| AAV-5: | (left) | 5′-GGAAAACTTGTCAGATTTT |
| | | GG-3′ |
| | (right) | 5′-GTCCAATCGATGCCACAAG |
| | | AGGCAGTATTTTACTGAC-3′ |
| AAV-6: | (left) | 5′-CTGGATGACTGTGTTTCTG |
| | | AGC-3′ |
| | (right) | 5′-GTCCAATCGATGCGAAGCG |
| | | CAACTAAGCAG-3′ |

1. An AAV helper plasmid comprising the following DNA sequences:
(a) i) the rep gene of AAV-2; and
ii) the cap gene of AAV-1, AAV-3, AAV-4, AAV-5 or AAV6; or
(b) the cap gene and the rep gene of AAV-1, AAV-3, AAV-4, AAV-5 or AAV-6 each; and
(c) all further helper virus DNA sequences necessary for forming AAV particles.

2. The AAV helper plasmid according to claim 1, wherein the helper virus DNA sequences originate from herpes virus.

3. The AAV helper virus according to claim 1, wherein the helper virus DNA sequences originate from adenovirus.

4. The AAV helper plasmid according to claim 3, wherein the adenovirus is adenovirus 5.

5. The AAV helper plasmid according to claim 4, wherein the helper virus DNA sequences are the Ad5 genes E2A, E4 and VA.

6. The AAV helper plasmid according to claim 5, which additionally contains an expression cassette for the expression of a fluorescent protein.

7. The AAV helper plasmid according to claim 6, wherein the fluorescent protein is the “red fluorescent” protein.

8. The AAV helper plasmid according to claim 7, wherein the fluorescent protein is functionally linked to an RSV promoter.

9. The AAV helper plasmid according to claim 8, which is the pDP1 with accession number DSM 14256, pDP3 with accession number DSM 14255, pDP4 with accession number DSM 14254, pDP5 with accession number DSM 14253 or pDP6 with accession number DSM 14252.

10. An AAV particle whose capsid coat is encoded by the AAV helper plasmid according to claim 1 and which contains an AAV expression vector.

11. The AAV particle according to claim 10, wherein the AAV expression vector comprises at least the following DNA sequences:
(a) the 5′ITR and 3′ITR of an AAV-2;
(b) a constitutive or inducible promoter active in mammals, and
(c) a polyadenylation signal.

12. A medicament containing an AAV helper plasmid according to claim 1 and a pharmaceutically compatible carrier.

13. A method for introducing an expression vector for gene therapy comprising introducing into a cell an AAV helper plasmid according to claim 1.

14. A mammalian cell containing the AAV particle according to claim 10.

15. The mammalian cell according to claim 14, which is an 293 cell.

16. A method of producing a pseudo-typed AAV particle, the method comprising transfecting mammalian cells with an AAV helper plasmid according to claim 1 and an AAV expression vector and culturing the cells a sufficient time and under conditions for isolating the AAV particle is isolated from the mammalian cells or the medium.

17. The AAV helper plasmid according to claim 3, which additionally contains an expression cassette for the expression of a fluorescent protein.

18. An AAV particle whose capsid coat is encoded by the AAV helper plasmid according to claim 9 and which contains an AAV expression vector.

19. A medicament containing an AAV helper plasmid according to claim 9 and a pharmaceutically compatible carrier.

20. A medicament containing an AAV particle according to claim 10 and a pharmaceutically compatible carrier.

21. A medicament containing an AAV particle according to claim 11 and a pharmaceutically compatible carrier.

22. A mammalian cell containing the AAV particle according to claim 11.

23. A mammalian cell containing the AAV particle according to claim 20.

24. A method for introducing an expression vector for gene therapy comprising:
introducing into a cell an AAV helper plasmid according to claim 9.

25. A method for introducing an expression vector for gene therapy comprising:
introducing into a cell an AAV particle according to claim 10.
